(19) United States
(12) Patent Application Publication
(10) Pub. No.: US 2021/0153892 A1
(43) Pub. Date: May 27, 2021

(54) EPIDURAL DELIVERY DEVICE

(71) Applicant: TAUMED R&D LTD, Ramat Gan (IL)

(72) Inventor: Matan SHABAT, Eilat (IL)

(21) Appl. No.: 17/256,214

(22) PCT Filed: Jun. 26, 2019
(86) PCT No.: PCT/IL2019/050710

§ 371 Date: Dec. 27, 2020

(30) Foreign Application Priority Data
Jun. 26, 2018 (IL) 260273

(51) Int. Cl. A61B 17/34; A61B 34/20

(52) U.S. Cl. CPC A61B 17/3401; A61B 17/3403; A61B 34/20; A61B 2017/00199; A61B 2017/3407; A61B 2017/3492; A61B 2034/2051; A61B 2017/347

(57) ABSTRACT

A device for safe epidural drug delivery configured to be used with standard epidural stylet and needle. The correct placement of an epidural needle and stylet within the epidural space is provided with the device comprising at least one position sensor for determining the relative position of both an epidural needle and stylet and relative to one another.

### FIELD OF THE INVENTION

The present invention relates to a drug delivery safety device and in particular, to such a device providing safe epidural drug delivery with standard epidural stylet and needle.

### BACKGROUND OF THE INVENTION

Epidural anesthesia is a technique to reach the state of anesthetization by injecting some sedative into the epidural space. Those sedative blocks the spinal neural transmission temperately, so this surgery is also called “epidural block”, and is often applied on surgical operations. For anesthesiologists, the most important issue in epidural anesthesia is whether the needle tip is accurately inserted into the correct position (epidural space). As shown in FIG. 1, epidural space is a small lumen located between Dural matter and ligamentum flavum that is hard to position and detect precisely, and its' lumen size varies with age and body shape. Normally, the epidural space in the adult lumbar spine is 2-8 mm (millimeters) deep, but in thorax it is only 1-5 mm, which means it is comparatively easy to cross it and accidentally puncture the dura with the needle. This may in turn cause the Post Dural Puncture Headache (PDPH) or severe spinal injury.

In the epidural use, the insertion of needles into the epidural space is often time a blind procedure requiring secondary indirect confirmation using a loss of resistance method, or direct confirmation by injection of dye and confirmatory X-ray. Insertion of the tip of an epidural needle into the epidural space without perforation of the dural sac requires significant expertise and training. If the epidural needle is not advanced sufficiently past ligamentum flavum, the epidural space is not reached. Alternatively, if the tip of the needle is advanced too far, the dural sac may be punctured resulting in leakage of spinal fluid. If a puncture is recognized, typically anesthesia is converted from epidural anesthesia to spinal anesthesia. If the puncture goes unrecognized, severe complications arising from overdose or excessive anesthetic solution in the subdural space may result.

At present, anesthesiologists identify the correct position according to their experience and subjective judgment. Traditionally, they identify epidural space by feeling the resistance of ligamentum flavum, but the resistance of ligamentum flavum varies with age, gender and body shape. For example, the tenacity of ligamentum flavum in older individuals is weak, and those in pregnant women and obese people also have weak resistance.

U.S. Publication No. 2009/0099501 to Chang et al., U.S. Publication No. 2014/0303494 to Janicki et al., both rely on optical means to correctly penetrate the epidural space. However, such optical systems utilize specialized optical based guidance systems require customized stylet and needles and special training.

Other epidural guidance systems rely on still more expensive and specialized medical imaging devices and techniques such as ultrasound that require additional training and specialization.

### SUMMARY OF THE INVENTION

The present invention overcomes the deficiencies of the background by providing a device for safely preforming an epidural anesthesia in a controlled manner that ensures appropriate penetration of the epidural space and avoiding mistaken penetration of the dura matter.

Embodiments of the present invention provide a device capable of sensing and indicating the instant the epidural space is penetrated therein avoiding over penetration of the epidural space.

Embodiments of the present invention further provide a device that utilizes a conventional (non-specialized) epidural stylet and needle wherein the device is capable of sensing and indicating the instant the epidural space is penetrated. Accordingly providing savings associated with the costs and time required for specialized training and equipment.

Embodiments of the present invention provides a device that may be functionally associated and/or coupled and/or affixed to at least a portion of a conventional (non-specialized) epidural stylet and needle wherein the device is capable of sensing and indicating the instant the epidural space is penetrated.

Embodiments of the present invention provides a device that may be integrated and/or associated with at least a portion of an epidural stylet and/or an epidural needle assembly wherein the device is capable of sensing and indicating the instant the epidural space is penetrated. In an optional embodiment the device may be coupled and/or integrated with a portion of the needle. In an optional embodiment the device may be coupled and/or integrated with a portion of the stylet.

In an optional embodiment the device may be provided from at least two or more portions wherein including a first portion that is coupled and/or integrated with an epidural stylet and a second portion is couple and/or integrated with an epidural needle. Optionally a third portion of the device may be associated over the epidural access site and/or penetration site.

In embodiments of the present invention provide a single use device for facilitating instantaneous identification of the epidural space during epidural penetration.

Embodiments of the present invention provide a device comprising a positioning sensor in the form of a mechanical or an optical position sensor.

Embodiments of the present invention provides a device capable of fixing and/or locking the position of the epidural needle within the penetration and/or delivery canal so as to prevent further advancement of the epidural needle and/or stylet.

In embodiments of the present invention, provides a device comprising a movement module utilized to facilitate safe advancement of an epidural stylet and needle so as to allow the device to sense and indicate the instant the epidural space is penetrated so as to prevent over penetration. Optionally the movement module may for example including but is not limited to: a motor, hydraulic motor, electromagnetic motor, piston, air piston, spring loaded movement, gear, linear spring, windings, electromagnet, the like or any combination thereof.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1A is a schematic cross section illustration showing the different tissue layers that need to be penetrated in order to reach the epidural space in an epidural procedure; and

FIG. 1B is a schematic illustration showing a standard epidural needle and stylet as is known in the art that may be utilized with the device of the present invention; and

FIG. 2A-D are schematic block diagrams of an exemplary device according to embodiments of the present invention; and

FIG. 3 is a schematic illustrative diagrams of an exemplary device according to an embodiments of the present invention; and

FIG. 4A-B are schematic flow charts depicting optional method of use of the device according to optional embodiments of the present invention.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
50 Epidural stylet;
52 Epidural syringe/needle;
100,101 positioning device;
102 Penetration/delivery Canal;
102L needle locking module;
103 housing;
104 Position sensor;
105 movement module;
106 Advancement Display/Indicator;
108 surface positioning layer;
110 electronics module;
150 positioning device

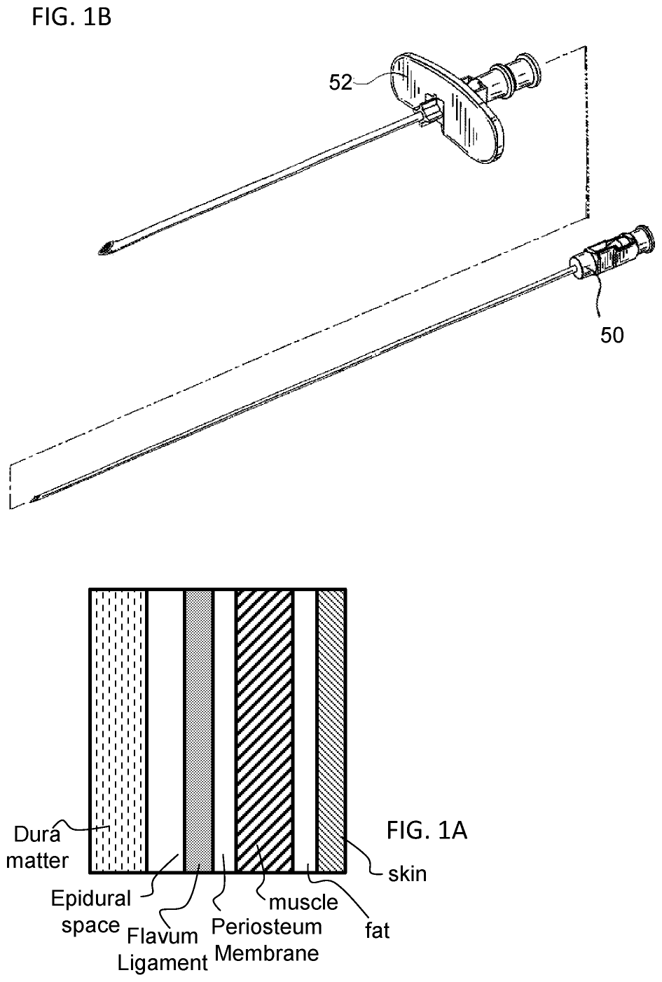

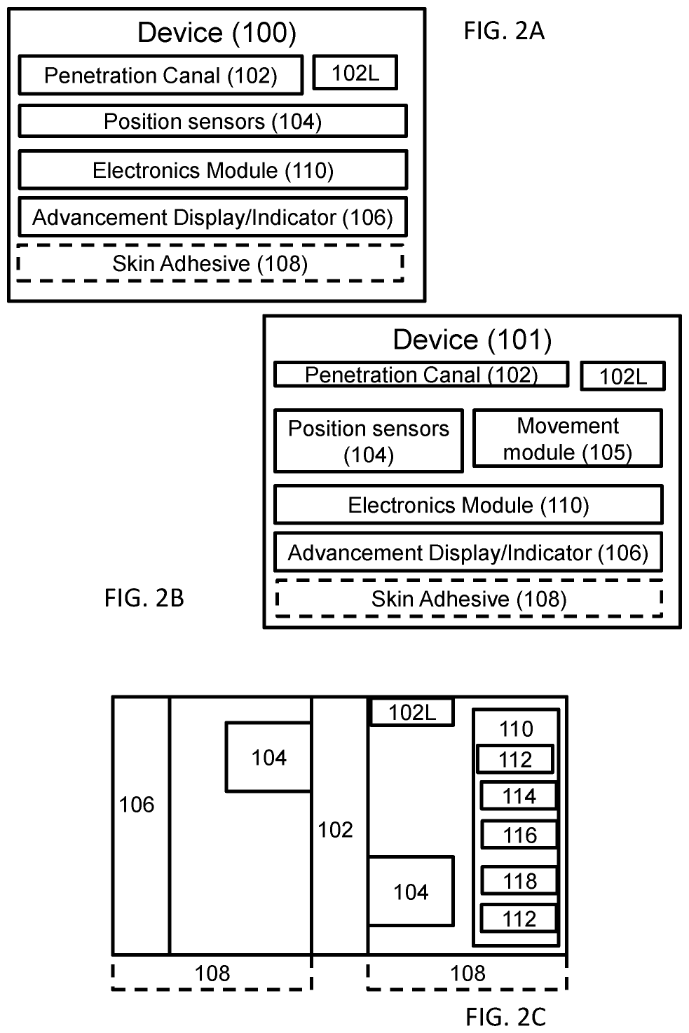

FIG. 1A shows a schematic cross section illustration of the different tissue layers that must be crossed from the surface of the skin toward the epidural space where an analgesic agent and/or medicament is to be delivered. Accordingly during an epidural procedure an epidural needle 52 and stylet 50, shown in FIG. 1B, must penetrate the various layers between the skin and the epidural space. During such procedure care must be taken not to cross the epidural space so as to no puncture the dura. Despite technological advancements at present the success of most epidural procedures are dependent on the skill level and experience of the practitioner and/or caregiver performing the delicate procedure.

The problem faced by practitioner is to ensure that they do not transcend and/or fully cross the width of the epidural space to the dura. Accordingly great care must be taken to ensure that the needle is not advanced too quickly as the width of the epidural space is on the order of 2 mm to about 8 mm. Accordingly due to the small width of the epidural space it is easy for a practitioner to mistakenly cross the epidural space.

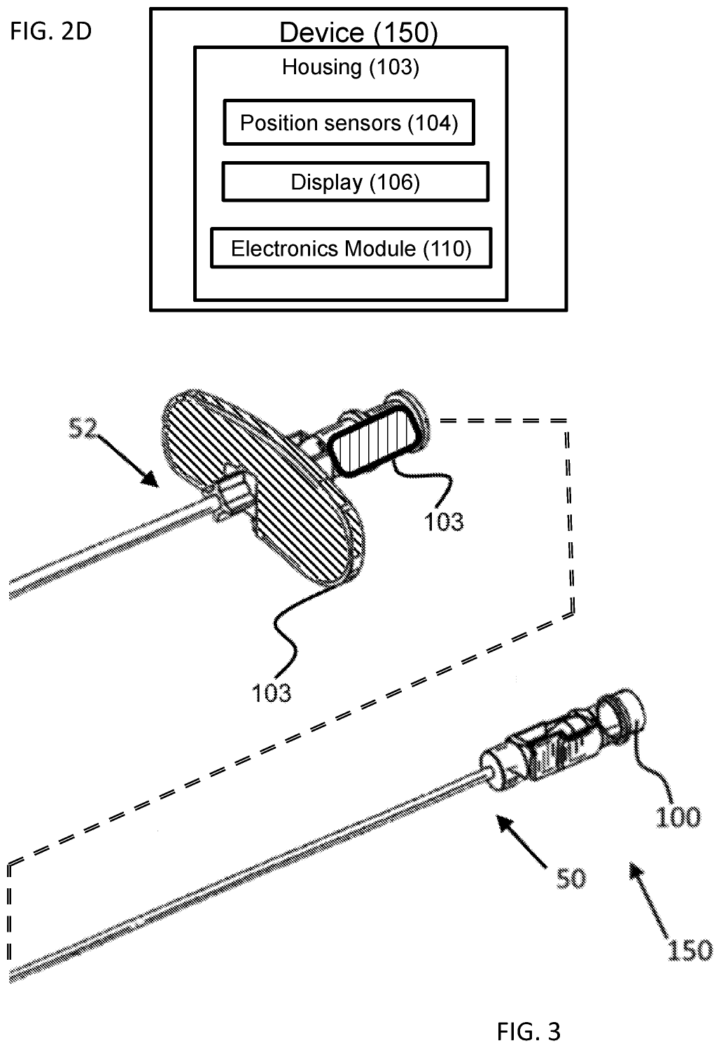

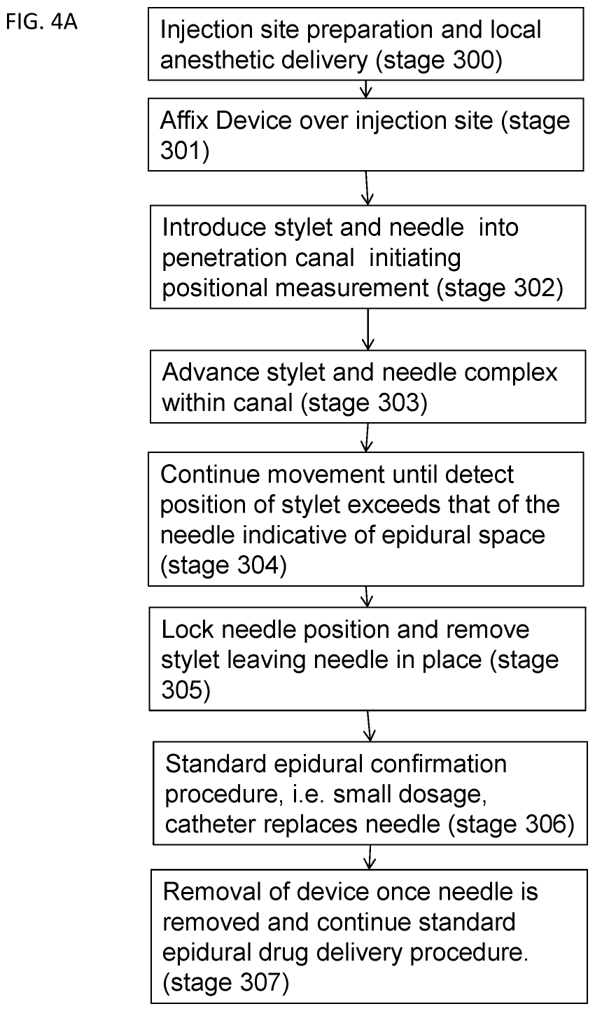

Embodiments of the present invention provides a device 100,101, 150 FIG. 2A-C and FIG. 3A-B, capable of detecting the instant the epidural spaces is accessed within a penetration depth of up to about 0.5 mm. In some embodiments device 100,101,150 is capable of detecting entry into the epidural space at a depth of from about 0.05 mm. In some embodiments device 100,101,150 may detect epidural space penetration at a depth of about 0.3 mm. In some embodiments device 100,101,150 may detect epidural space penetration at a depth of about 0.1 mm.

Accordingly, in detecting the depth of penetration at a penetration distance of from about 0.05 mm and up to about 0.5 mm provides a practitioner with an accurate and substantially instantaneous and/or real time indication when the epidural space has been reached therein allowing them to stop needle penetration.

A first embodiment of the present invention is shown in FIG. 2A showing a schematic block diagram of epidural delivery device 100. Device 100 is configured to be used with a standard epidural stylet 50 and needle 52 as are known in the art, as shown in FIG. 1B.

Device 100 comprises a penetration canal and/or delivery canal 102, at least one position sensor 104, an advancement display 106, and an electronics module 110. In some embodiments of device 100 may optionally further comprise a surface positioning layer 108 for example including but not limited to a skin surface adhesive layer.

Penetration canal 102 provides for receiving the epidural stylet 50 and needle 52 at a distal end so as to allow advancement of the stylet and needle 50,52 toward a proximal end, wherein the proximal end is defined along the patient's skin side overlying the intended injection and/or introduction and/or puncture site. Canal 102 therein allows the epidural stylet and needle complex to penetrate the body in a controlled manner.

In some embodiments the angle of canal 102 relative to surface of the skin may be pre-set and/or predefined. For example, the canal angle with respect to the skin surface at the injection site may be perpendicular 90 degrees, 70 degrees, 80 degrees, 60 degrees, 45 degrees, 30 degrees, 25 degrees, 15 degrees or the like preset angle.

In some embodiments the angle formed by canal 102 may be configurable and/or controlled and/or adjusted on site by a practitioner, at the time of use.

In embodiments device 100 comprises at least one position sensor 104, in some embodiments device 100 may be provided with at least two position sensors 104. Position sensor(s) 104 provides for sensing and/or determining the relative position of each of the stylet 50 and needle 52 from the moment they enter canal 102. Preferably the relative advancement of the epidural stylet and needle are monitored by sensors 104 with the further assistance of electronics module 110. Preferably sensor 104 provides an indication of the depth of penetration of stylet 50 and needle 52 in a continuous manner within canal 102 and tissue depth shown in FIG. 1A.

In embodiments sensor 104 and/or associated electronics module 110 or a combination thereof will determine the relative positions of style 50 relative to needle 52; in particular to identify the instance and/or conditions where the distal end of stylet 50 is advanced past the distal end of needle 52. Such a configuration is indicative of entry and/or penetration into the epidural space.

The epidural space is at a negative pressure and therefore once it is penetrated it would cause the distal tip of the stylet to advance past the distal tip of the needle as the stylet is more susceptive and or responsive to the change in pressure. Sensor 104 is configured to identify this situation to indicate to a practitioner that the epidural space has been penetrated. Preferably this is indicated with at least one of a visual, tactile and/or audible cue that is preferably communicated to display 106.

In embodiments position sensor 104 may be provided in optional forms for example including but not limited to optical sensors, mechanical sensors, electromagnetic sensor, induction sensor, magnetic based sensors or the like sensor provided for determining and the relative position of stylet and needle within delivery and/or penetration canal 102.

Preferably once the epidural space has been identified the needle 52 may be locked into position within canal 102 via a locking module 102L.

In an optional embodiment device 100 may further comprise a surface positioning layer 108 for example in the form of a skin adhesive layer 108 provided to affix and/or couple device 100 to the skin of the patient.

Locking module 102L provides for locking the position of epidural needle 52 within the penetration canal 102 so as to prevent accidental further advancement of the needle 52 while allowing the removal of stylet 50 enabling the additional steps required to complete the epidural anesthetic delivery as is known in the art.

In embodiments display 106 may be provided in optional forms for example including but not limited to an indicator, a plurality of indicators, an alphanumeric display, touch screen, LED display, the like or any combination thereof. Preferably display 106 provides an indication of the epidural stylet and needle complex position relative to the penetration canal 102. In some embodiments display 106 provides a user with a visible display and/or cue of the relative penetration depth of the epidural stylet and syringe. In embodiments the display may be indicated by an alphanumeric indication of penetration depth indicative of the instantaneous penetration length of the syringe and stylet following coupling with device 100,101 by way of insertion into delivery canal 102.

FIG. 2B shows an embodiment of the present invention showing epidural delivery device 101. Device 101 is similar to device 100 described with respect to FIG. 2A, however, further comprising a movement module 105. Movement module 105 provides for controllably advancing epidural stylet 50 and epidural needle 52 within penetration canal 102 toward the targeted epidural space. Optionally movement module 105 may be rendered operational in various manners for example including but not limited to an automated movement, semi-automatic movement, and/or a manually operated module.

In embodiments movement module 105 may be provided in optional forms for example including but not limited to a motors, hydraulic motor, piston, air piston, spring loaded movement, gear, linear spring, windings, electromagnet, piezoelectric actuator, the like or any combination thereof.

In embodiments movement module 105 may be utilized to lock the position of epidural needle 52 within the penetration canal so as to lock the needle's position preventing additional advancement of the epidural needle 52.

In some embodiments movement module 105 may be disposed along a portion of the penetration canal 102.

In some embodiments movement module 105 may be disposed along portion of a stylet 50 of the epidural needle 52.

In an optional embodiment of the epidural needle 52 may be locked into position with a locking module 102L that is associated with penetration canal 102. Optionally locking module 102L may be disposed along any portion of canal 102 preferably along its length. More preferably locking module is disposed along an upper and/or distal portion of canal 102.

In some embodiments locking module 102L may be provided in various optional forms for example including or comprising but not limited to at least one or more of: a mechanical stopper, a gripping mechanism, a locking pin, male/female couplers, corresponding pin and recess, clamp, notch lock clamp, any combination thereof or the like.

In embodiments display 106 may be provided in optional forms for example including but not limited to an indicator, a plurality of indicators, an alphanumeric display, touch screen, LED display, the like or any combination thereof. Preferably advancement display 106 provides an indication of the epidural stylet and needle complex position relative to the penetration canal 102. More preferably display 106 provides a user with a visible display and/or cue of the relative penetration depth of the epidural stylet and syringe. In embodiments the display may be indicated by an alphanumeric indication of penetration depth indicative of the instantaneous penetration length of the syringe and stylet following coupling with device 100,101 by way of insertion into delivery canal 102.

In some embodiments display 106 may additionally comprise an audible cue so as to generate and provide an audible indication to a user.

In embodiments depicted in FIG. 2A-B device 100,101,150 preferably comprises an electronics module 110. Electronics module 110 comprises a plurality of optional sub-modules for example including but not limited to a power supply sub-module 112, controller and/or processor sub-module 114, and memory sub-module 118. Optionally electronics module 110 may further comprise a communication module sub-module 116.

In embodiments processor sub-module 114 provides the necessary processing hardware and/or software necessary to render device 101,100,150 functional.

In embodiments power sub-module 112 provides the necessary hardware and/or software to power device 100,101,150 therein rendering device 100,101,150 operational.

In embodiments communication sub-module 116 provides the necessary hardware and/or software to facilitate communication for device 100,101,150 with optional auxiliary devices (not shown).

In embodiments memory sub-module 118 provides the necessary hardware and/or software to facilitate operations of device 100,101,150.

In embodiments controller and/or processor 114 may provide for controlling any portion of device 100,101,150 and in particular proximity sensor 104 and display 106. Wherein processor 114 is utilized to determine the relative position of the epidural stylet 50 and syringe 52 within the penetration canal 102 with the use of position sensors 104.

In one aspect of the invention device 101 controller 114 may further provide for controlling movement module 105.

In some embodiments power supply 112 may be utilized to power device 100,101,150 therein rendering device 101,100,150 functional. Power supply 112 may for example be provided in the form for example including but not limited to photo-galvanic cells, battery, rechargeable battery, disposable batteries, capacitors, super capacitors, or a mains power supply line, the like power source or any combination thereof.

In some embodiments electronics module 110 may further comprise a communications sub-module 116 that may be utilize to provide device 100,101,150 with communication capabilities. For example communication sub-module 116 may provide for communication with auxiliary devices and or systems by utilizing various communication protocols for example including but not limited to wireless communication protocols, cellular communication, wired communication, near field communication, Bluetooth, optical communication, the like and/or any combination thereof. In some embodiments device 100,101,150 may be in communication with an auxiliary device for example including but is not limited to an imaging device, ultrasound, X-ray, MRI, functional MRI (fMRI), CT, computer, server, smartphone, mobile telephone, portable device comprising a processing and communication capabilities, healthcare provider computerized system, medical device console, other devices, the like or any combination thereof.

In embodiments device 100,101,150 may be in communication with or functionally associated with at least one or more auxiliary devices for communicating positional data and/or an alarm state.

FIG. 2C provides a schematic illustration of the epidural device 100,101 as previously described, specifically showing an optional relative disposition of the penetration canal 102 and sensors 104.

Now referring to FIG. 2D and FIG. 3, shows an embodiment of the present invention wherein device 100,101 as previously described, may be realized in the form of a needle and/or stylet positioning device 150 wherein positioning device 100,101 are disposed and/or integrated along at least a portion of a stylet 50 and/or needle 52 by way of using a housing 103 configured to fit over at least a portion of needle 52 and/or stylet 50, for example as shown. In some embodiments, positioning device 100 having a specialized housing 103 that is configured to be disposed along a proximal portion of stylet 50 in and around the hub for placement in epidural needle 52. Device 150 preferably provides for using a stylet and catheter in the standard manner as is known by current practice.

In embodiments device 150, shown in FIG. 2D, comprises housing 103, position sensor module 104 as previously described, indicator 106 as previously described, and electronics module 110 as previously described.

Housing 103 provided for associating and/or integrating with at least one or both of stylet 50 and/or needle 52.

In some embodiments housing of device 103 of device 150 may be provided as a single housing and/or a multi-unit housing. A multi-unit housing may be realized by having portions of device 100 rendered along different portions of needle 52 and/or stylet 50.

In some embodiments device 150 may be realized as an add-on device wherein housing 103 is associated and/or coupled onto an “off the self” standard stylet and needle.

In some embodiments device 150 may be realized as a device integrated and/or prefabricated with a dedicated stylet and needle.

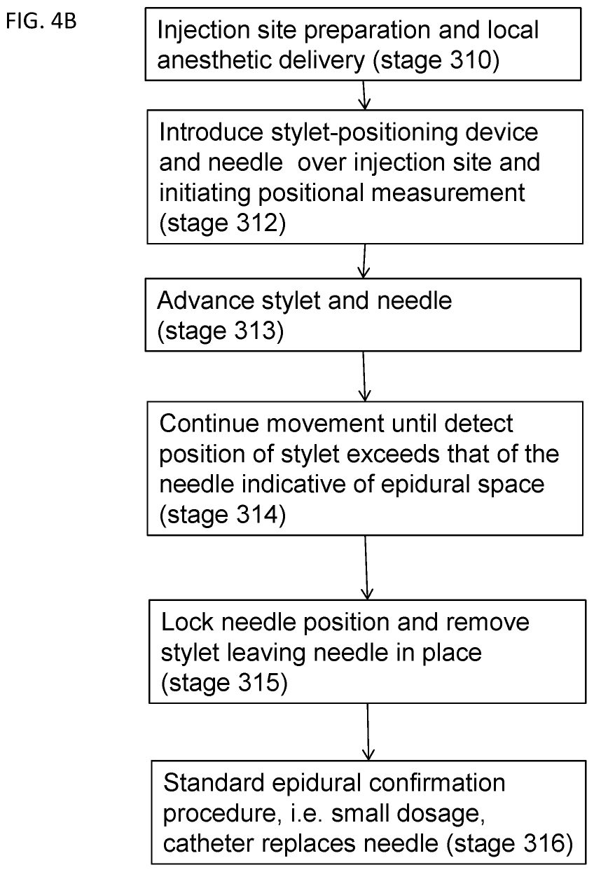

FIG. 4A shows a flow chart of the method of use of device 100,101 according to embodiments of the present invention.

Initially in stage 300 the injection site preparation is undertaken according to standard epidural drug delivery protocol, where the injection site is selected and local anesthesia is applied.

Next in stage 301 device 100,101 is associated over the intended injection site such that canal 102 is directly overlying the injection site. Optionally device 100,101 may be affixed to the skin surface with an optional adhesive layer 108.

Next in stage 302 the epidural procedure continues by introducing the stylet 50 and needle 52 into canal 102. The introduction is registered by sensor 104 and electronics module 110, providing a zeroing reference point from which future depth measurements will be determined.

Next in stage 303, the stylet and needle are advanced through the different layers, skin, fat, muscle, periosteum membrane and Flavum Ligament. In some embodiments with device 101 featuring automated movement module 105 the stylet and needle are automatically and controllably advanced.

Next in stage 304, the stylet and needle are continually advanced through the different issue layers until the epidural space is reached wherein the negative pressure of the epidural space cause misalignment of the distal end tips of stylet 50 and needle 52. Accordingly, once stylet 50 and needle 52 are no longer in alignment wherein the position of the distal end tip of stylet 50 has penetrated the epidural space causing the distal tips not to be aligned. The threshold distance between the tips is detected with position sensor 104 and/or electronic circuitry 110. Accordingly, sensors 104 are configured to detect when stylet 50 progresses an additional threshold distance from about 0.05 mm and up to about 0.5 mm ahead of needle 52 toward the dura. The misalignment between the distal tips of stylet and needle is indicative that the epidural space has been reached.

Next in stage 305 the position of needle is locked preferably with locking module 102 and stylet is removed.

Next in stage 306 standard epidural procedures are undertaken to ensure that correct placement and dosage is tested and provided as is the standard procedure that includes catheter placement through needle 52 and removal of the needle. Once needle is removed device 100,101 may be removed, provided in final stage 307, and the standard epidural course of action is continued.

FIG. 4B shows a flow chart of the method of use of device 150, FIG. 2D, FIG. 3) according to an optional embodiments of the present invention. In an optional preparatory stage (not shown in FIG. 4B) according to some embodiments of device 150, housing 103 of device 150 is associated with a stylet and needle so as to form a functional ready to use device 150. Initially in stage 310 the injection site preparation is undertaken according to standard epidural drug delivery protocol, where the injection site is selected and local anesthesia is applied.

Next in stage 312 the epidural procedure continues by introducing the device 150 and needle 52 over injection site. This stage is accomplished to register the relative positions of sensor 104 and electronics module 110, providing a zeroing reference point from which future depth measurements will be determined.

Next in stage 313, the stylet and needle are advanced through the different layers, skin, fat, muscle, periosteum membrane and Flavum Ligament. In some embodiments with device 101 featuring automated movement module 105 the stylet and needle are automatically and controllably advanced.

Next in stage 314, the stylet and needle are continually advanced through the different issue layers until the epidural space is reached wherein the negative pressure of the epidural space cause misalignment of the distal end tips of stylet 50 and needle 52. Accordingly, stylet 50 and needle 52 are no longer in alignment wherein the position of the distal end tip of stylet 50 has penetrated the epidural space causing the distal tips not to be aligned. The threshold distance between the tips is detected with position sensor 104 and/or electronic circuitry 110. Accordingly, sensors 104 are configured to detect when stylet 50 progresses an additional threshold distance from about 0.05 mm and up to about 0.5 mm ahead of needle 52 toward the dura. The misalignment between the distal tips of stylet and needle is indicative of that the epidural space has been reached.

Next in stage 315 the position of needle is fixed.

Next in stage 316 standard epidural procedures are undertaken to ensure that correct placement and dosage is tested and provided as is the standard procedure that includes catheter placement through needle 52 and removal of the needle.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are no way limiting on the scope of the claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1) A device for correct placement of an epidural needle and stylet within the epidural space, the device including at least one position sensor configured for determine the relative position of both of said epidural needle and stylet relative to one another, an electronic module functionally associated with said at least one position sensor; and a penetration canal for receiving an assembly of said epidural needle and said stylet; and wherein said at least one position sensor is provided for measuring the relative position of said needle and stylet within said canal.

2) (canceled)

3) The device of claim 1 comprising at least two position sensors.

4) The device of claim 1 further comprising a display.

5) The device of claim 1 further comprising a surface positioning layer capable of coupling the device over an injection site.

6) The device of claim 5 wherein said surface positioning layer is provided in the form of a skin surface adhesive.

7) The device of claim 1 further comprising a locking module for fixing the position of the stylet and needle assembly within said delivery canal.

8) The device of claim 1 wherein said canal is provided configured to have a prefixed angle relative to the treated surface.

9) The device of claim 1 wherein the angle assumed by said canal is adjustable.

10) The device of claim 9 wherein said canal angle may be set from 30 degrees up to about 90 degrees relative to the injection site surface.

11) The device of claim 1 wherein said at least one position sensor is configured to detect a threshold distance correlated to the difference in the relative position of the distal ends of the epidural syringe and epidural stylet.

12) The device of claim 11 wherein the threshold distance is from 0.05 mm up to 0.5 mm.

13) The device of claim 11 wherein the threshold distance is 0.1 mm.

14) The device of claim 11 wherein the threshold distance is 0.2 mm.

15) The device of claim 11 wherein the threshold distance is 0.3 mm.

16) The device of claim 11 wherein the threshold distance is 0.4 mm.

17) The device of claim 1 further comprising a movement module for controlling the advancement of the needle and stylet assembly disposed within the penetration canal.

18) The device of claim 17 wherein said movement module at least one or more selected from: a motor, hydraulic motor, piston, air piston, spring loaded movement, gear, linear spring, windings, electromagnet, piezoelectric actuator, or any combination thereof.

19) The device of claim 1 having a housing configured to be coupled with at least a portion of one of: a stylet, a needle, or a combination thereof.

20) The device of claim 1 provided with a multi-unit housing formed from at least two portions.

21) The device of claim 19 wherein said housing is a multi-unit housing formed from at least two portions.
